(19) United States
(12) Patent Application Publication
(10) Pub. No.: US 2016/0008492 A1
(43) Pub. Date: Jan. 14, 2016

(54) COMPOSITIONS OF SACCHARIDE COATED NANOPARTICLES AND USES

(71) Applicant: EMORY UNIVERSITY, Atlanta, GA (US)

(72) Inventors: Hui Mao, Johns Creek, GA (US); Jing Huang, Decatur, GA (US)

(21) Appl. No.: 14/797,775

(22) Filed: Jul. 13, 2015

Related U.S. Application Data
(60) Provisional application No. 62/024,151, filed on Jul. 14, 2014.

(51) Int. Cl. A61K 49/18; A61K 49/12

(52) U.S. Cl. CPC A61K 49/1824; A61K 49/126

(57) ABSTRACT

This disclosure relates to nanoparticles of metals, alloys, metal oxides, and multi-metallic oxides comprising oligosaccharide coatings and uses such as in molecular imaging. Typically, the nanoparticles comprise iron oxide. In certain embodiments, the disclosure relates to compositions comprising iron oxide nanoparticles comprising a saccharide coating of less than 5 nm. In certain embodiments, the average core size is below 5 nm with highly uniform core size and a hydrodynamic size of not greater than 10 nm.

### CROSS REFERENCE TO RELATED APPLICATIONS

This Application claims priority to U.S. Provisional Application No. 62/024,151 filed Jul. 14, 2014, which application is hereby incorporated by reference in its entirety.

### STATEMENT REGARDING FEDERALLY FUNDED RESEARCH

This invention was made with government support under Grants Nos. R01CA154846 and U01CA151810-02 awarded by National Institutes of Health. The government has certain rights in the invention.

### BACKGROUND

Feridex® and Resovist® are iron oxide nanoparticles (IONPs) formulated with dextran coatings and have been approved for clinical use as magnetic resonance imaging (MRI) contrast agents. See Reddy et al., Chem Rev, 2012, 112 (11):5818-78. After systemic administration both are trapped in the organs of the reticuloendothelial system (RES) and take several weeks to be degraded and cleared from the body. Slow clearance causes concern about long term side effects.

Thermal decomposition of iron salts of long chain carboxylic acids is a method of preparing IONPs of controlled sizes. Park et al. Nat Mater, 2004, 3 (12):891-5 and Yu et al. ChemComm, 2004, 2306-2308. The resultant hydrocarbon coated IONPs have a tendency to aggregate in aqueous media which is typically remedied by inserting or substituting hydrophilic coatings such as dextran, carboxydextran, polyethylene glycol (PEG), or poly(methacrylic acid) (PMAA). See U.S. Pat. Nos. 9,028,875, 7,811,545, and 5,770,172.

Kim et al. report synthesis of small-sized iron oxide nanoparticles for magnetic resonance imaging contrast agents. J Am Chem Soc, 2011, 133 (32):12624-31. However, at single digit nanoscale core nanoparticle sizes hydrophilic surface coatings are not typically uniform which may cause inconsistent MRI contrast enhancement and signal quantification. Thus, there is a need to identify improvements.

Smolensky et al. report the effect of particle size and shape on the magnetism and relaxivity of iron oxide nanoparticle contrast agents. J Mater Chem B Mater Biol Med, 2013, 1 (22):2818-2828.

References cited herein are not an admission of prior art.

### SUMMARY

This disclosure relates to nanoparticles of metals, metal alloys, metal oxides, and multi-metallic oxides comprising oligosaccharide coatings and uses such as in molecular imaging. Typically, the nanoparticles comprise uniform core size iron oxide. In certain embodiments, the disclosure relates to compositions comprising iron oxide nanoparticles comprising a saccharide coating of less than 5 nm. In certain embodiments, the average core size is below 5 nm with a hydrodynamic size of not greater than about 10 nm.

In certain embodiments, the average core size is below about 5 nm, e.g., an average core size is 3.5 nm or 4.8 nm, with a hydrodynamic size of not greater than about 10 nm, e.g., about 7.3 nm or 9.5 nm respectively.

In certain embodiments, the disclosure relates to compositions comprising nanoparticles of metals, metal oxides and multi-metallic oxides or quantum dot of a size of between 3 nm to 100 nm comprising a saccharide coating of less than 5 nm.

In certain embodiments, nanoparticles with the saccharide coating are made by the process of heating the nanoparticles in a mono-, di- tri-saccharide, or larger oligosaccharide in dimethylformamide. In certain embodiments, the mono-saccharide is a hexose sugar such as glucose, allose, altrose, mannose, gluose, iodose, galactose talose, or a pentose sugar such as fructose, ribose, arabinose, xylose, and lyxose. Other contemplated monosaccharides are galactosamine, glucosamine, sialic acid, and N-acetylglucoseamine.

In certain embodiments, the di-saccharide is selected from sucrose, lactulose, lactose, maltose, trehalose, cellobiose, chitobiose, kojibiose, nigerose, isomaltose, trehalose, sophorose, laminarbiose, gentiobiose, turanose, maltulose, palatinose, gentiobiulose, mannobiose, melibiose, melibiulose, rutinose, rutinulose, and xylobiose.

In certain embodiments, the tri-saccharide is selected from isomaltostriose, nigerotriose, maltotriose, melezitose, maltotriulose, raffinose, and kestose.

In certain embodiments, the heating is at 120 degrees Celsius for 2.5 hours. In certain embodiments, the heating is above 100 degrees Celsius for more than one hour. In certain embodiments, the heating is below 150 degrees Celsius for less than three hours.

In certain embodiments, the disclosure relates to nanoparticles or nanoparticles with the saccharide coating having a core made by the process of mixing a metal salt oleate such as iron (III) oleate with 1-octadecene and heating the mixture at a rate of 0.6 degrees Celsius per second until reaching 320 degrees Celsius, holding for 5 minutes at 320 degrees Celsius, and thereafter cooling.

In certain embodiments, nanoparticles or nanoparticles with the saccharide coating have core made by the process of mixing a metal salt of oleate such as iron (III) oleate with 1-octadecene and heating the mixture at a rate of 0.01 to 1.0 or 0.5-0.7 degrees Celsius per second until reaching 300 to 320 or 318 to 322 degrees Celsius, holding for 2 to 30 or 4 to 6 minutes at 300 to 320 or 318 to 322 degrees Celsius, and thereafter cooling.

In certain embodiments, the metal or iron (III) oleate and 1-octadecene are in a 1:1 mixture by volume mixture. In certain embodiments the iron (III) oleate and 1-octadecene are in a 0.5-5 to 5-0.5 or 0.8-1.2 to 1.2-0.8 by volume mixture.

In certain embodiments, the disclosure relates to nanoparticles or nanoparticles with the saccharide coating as such that about 12% of the nanoparticles have a core size of 3.1 nanometers, about 29% of the nanoparticles have a core size of 3.3 nanometer and about 24% of the nanoparticles have a core size of 3.6 nanometers and about 16% of the nanoparticles have a core size of 3.9 nanometer.

In certain embodiments, the disclosure relates to nanoparticles or nanoparticles with the saccharide coating as such that 11-14% of the nanoparticles have a core size of 2.9-3.2 nanometers, 27-30% of the nanoparticles have a core size of 3.2-3.5 nanometer and 23-25% of the nanoparticles have a core size of 3.5-3.8 nanometers and 15 to 18% of the nanoparticles have a core size of 3.8-4.1 nanometer.

In certain embodiments, the disclosure relates to nanoparticles or nanoparticles with the saccharide coating wherein the core comprises iron oxide, Mn, Gd, or combinations such as iron oxide and Gd, or iron oxide and Mn. With regard to any of the embodiments disclosed herein the nanoparticles comprising iron oxide are contemplated to include cores comprising combinations such as iron oxide and Gd, or iron oxide and Mn.

In certain embodiments, the disclosure relates to methods of magnetic resonance imaging comprising: a) administering nanoparticles as disclosed herein to a subject, b) applying a magnetic field and radio frequency energy to a region of the subject to be imaged, c) obtaining a hyperintense magnetic resonance signal image data set, and d) displaying the image data set; wherein the magnetic resonance signal image data set is associated with distribution of the iron oxide nanoparticles in the region. In certain embodiments, the methods optionally further comprises administering a contrast agent comprising Gd³⁺. In certain embodiments, the iron oxide nanoparticles comprise Gd.

### BRIEF DESCRIPTION OF THE FIGURES

FIG. 1 shows a low magnification TEM image of hydrophobic IONPs sized in 3.5 nm (IO-3). The inset of is the size distribution after measured 100 nanoparticles.

FIG. 2A Powder XRD pattern spectra of IO-3 and SIO-3.

FIG. 2B shows FTIR spectra of IO-3 and SIO-3.

FIG. 3A shows T₁-weighted MR images of SIO solutions with different concentrations.

FIG. 3B T₂-weighted MR images of SIO solutions with different concentrations.

FIG. 3C shows r₁ value changes with nanoparticle size.

FIG. 3D shows r₂ value changes with nanoparticle size.

FIG. 4A shows the maximum SI of each SIO solutions related to the r₁/r₂ ratio.

FIG. 4B shows r₁/r₂ ratio changes with hydrodynamic sizes.

FIG. 5A shows the size distribution of hydrophobic IONPs (SIO-3) after modified with oligosaccharides.

FIG. 5B shows the size distribution of hydrophobic IONPs (SIO-5) after modified with oligosaccharides.

FIG. 6A shows data indicating the dual contrast effect of SIO-3 improves the sensitivity and image clarity for visualizing the morphology of the liver parenchyma and structure of hepatic vasculature. Contrast changes between liver parenchyma and vessels.

FIG. 6B shows calculated complex magnetic susceptibility spectra of SIO3, SIO10 and SIO-20 recorded at 298K. SIO-20 has the highest sensitivity to an external magnetic field, which is responsible for its highest transverse relaxivity r₂ measured by MRI.

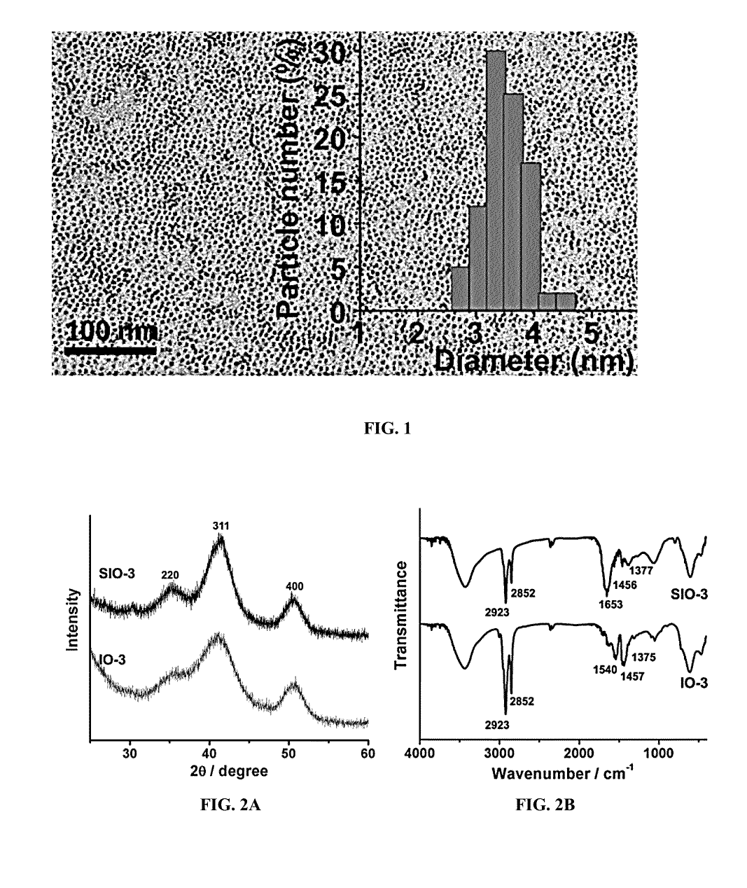

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used herein, “subject” refers to any animal, preferably a human patient, livestock, or domestic pet. According to a further embodiment of the disclosure, the methods discussed herein are applied as a screening technique to patients having no known or suspected disease or abnormality.

As used herein, the term “about” refers to a numerical value that can vary by 10 or 20%.

### Nanoparticles of Metals, Metal Oxides and Multi-Metallic Oxides

Processes for producing nanoparticles are reported in U.S. Pat. Nos. 9,028,875, 7,811,545, and 5,770,172. Using process provided therein, herein, or other known methods, one can substitute iron salts with other salts containing metal ions, e.g., metal salts comprising metal ions selected from the group consisting of Fe, Co, Ti, V, Cr, Mn, Ni, Cu, Zn, Y, Zr, Mo, Ru, Rh, Pd, Ag, Cd, Ce, Pt, Au, Ba, Sr, Pb, Hg, Al, Ga, In, Sn Ge or mixtures thereof.

Hydrophobic iron oxide nanoparticles may be synthesized by thermo-decomposition of a metal oleate. Providing a 1:3 molar mixture of the metal salt to sodium oleate may be dissolved in distilled water, hexane and ethanol with mixing, e.g., until resulting in colored hexane layer. The hexane layer may be used as the metal source for thermo-decomposition wherein the metal oleate is mixed with 1-octadecene. Thereafter the reaction mixture is heated under conditions to provide metal, metal oxides, and multi-metallic oxides, e.g., typically 300-350 C for 20-90 minutes.

Particles made by these processes can be coated with an oligosaccharide using the processes reported herein for coating the IONPs.

### Non-Hydrothermal Synthesis of Oligosaccharides Coated Sub-5 nm Magnetic Iron Oxide Nanoparticles with Dual MRI Contrast Enhancement Effect

Stable and biocompatible sub-5 nm magnetic iron oxide nanoparticles (IONPs) coated with oligosaccharides (SIO) have been developed for improving contrast enhanced MRI. The stability of such sub-5 nm IONPs is achieved by in-situ polymerization of glucose on the nanoparticle surface. The resulted ultrafine SIOs exhibit excellent colloidal properties in physiological medium and bright T₁ MRI contrast enhancement, providing a potential blood pool MRI contrast agents with longer blood half-time than small molecule contrast agents. Furthermore, T₂-to-T₁ contrast switching observed in liver imaging provides a potential application of simultaneous imaging of liver tumor morphology and vasculature with the demonstrated “dual” contrast. More importantly, sub-5 nm SIO showed the faster degradation and clearance by RES than the SPIO with the larger size, thus, may potentially address the toxicity concerns.

IONPs with different diameters were prepared by thermal decomposition of ferric oleate through adjusting decomposition conditions. The hydrophobic IONPs were uniform with diameters of 3.5 (IO-3), 4.8 (IO-5), 9.9 (IO-10), 15.6 (IO-15), and 19.9 nm (IO-20) respectively, as revealed TEM images. The hydrophobic IO nanoparticles were mixed with glucose solution in DMF, and heated to allow the in situ-polymerization of glucose on the nanoparticle surface. A thin oligosaccharides coating layer was formed, rendering water soluble nanoparticles. The core sizes showed no significant changes before and after the surface modification. To evaluate the hydrodynamic diameters of these oligosaccharides coated IONPs in aqueous solution, DLS measurement were performed. The hydrodynamic sizes are 7.3, 9.5, 11.5, 15.7, 20.9 nm for SIO-3, 5, 10, 15, 20, respectively, which are slightly larger than the TEM core sizes due to the addition of the hydrophilic oligosaccharide coating layers. The hydrodynamic size of 7.3-nm measured in SIO-3 suggests the thinnest coating layer among those IONPs with core size below 5 nm, which may play the significant role in preserving the T₁ contrast enhancing effect due to less restraints in water exchange between inner and outer layers. Moreover, the small hydrodynamic size indicates the single dispersion of SIO-3 in the aqueous solution preventing the T₂ effect caused by the aggregation

The powder X-ray diffraction (XRD) patterns of IO-3 and SIO-3 are shown in FIG. 2A. Broadened diffraction peaks were observed for both samples due to the ultrafine nano-sized crystals. The broadened diffraction peaks became clearer after applying the coating, due to the rearrangement of the canted surface during the heating process. However, the grain size changed little according to the half width of the diffraction peaks. Both of the XRD peaks of IONPs before and after coating were assigned to the spinal magnetite or maghemite structure. The formation of oligosaccharide coating on the surface of IONPs was further confirmed by Fourier-transform infrared spectroscopy (FTIR) (FIG. 2B). The characteristic bands of oleic acid, including C—H stretching (2923, 2852 cm−1), CH2 bending (1457, 1375 cm−1) and C[double bond, length as m-dash]O stretching (1540 cm−1), became weakened after being replaced by oligosaccharides on the surface. The emerged sharp C[double bond, length as m-dash]C band at 1653 cm−1 indicated the presence of aromatic structures of oligosaccharides on the nanoparticle surface.

It should be noted that the temperature used for in situ polymerization of glucose on the surface of nanoparticles is much lower (about 120° C.) than that of the established hydrothermal methods used to synthesize carbonized materials from glucose. When the formation of oligosaccharides was monitored by UV and fluorescent spectroscopy, a turquoise fluorescent signal with UV excitation at λ=365 nm was observed after 0.5 h reaction. This signal can be ascribed to the aromatic groups derived from the intermolecular dehydration and aldol condensation during glycosylation. At this time, oleic acid capped IONPs began to transfer into the aqueous phase as the formation of oligosaccharide coating took place. However, at this early stage, the hydrophilic oligosaccharides were insufficient to stabilize the nanoparticles in the aqueous solution, resulting in a light yellow turbid suspension. In order to keep the small size of the whole nanoparticle, oligosaccharide coating was controlled to be minimal, but sufficient to stabilize the nanoparticles. When the IONPs were transferred completely and dispersed into aqueous solution (i.e. reaction time is 2.0 h, yielded brownish transparent solution), the reaction was terminated immediately. Parallel experiments indicated the pivotal role of DMF solvent in the formation of oligosaccharide coating, together with the possible catalytic effect from cationic iron. DMF provides an alkaline condition for the glycosylation and facilitates the reaction by absorbing water molecules produced during the polymerization. Evidently, glycosylation under the same reaction conditions, but in different solvents, e.g., octadecene (ODE), diethylene glycol (DEG), and dimethyl sulfoxide (DMSO), did not yield such sufficient oligosaccharides.

### Methods of Use

In certain embodiments, the disclosure contemplates using nanoparticles disclosed herein for imaging. In certain embodiments, the disclosure relates to methods of imaging comprising: a) administering nanoparticles as in disclosed herein to a subject, b) applying electromagnetic radiation to a region of the subject to be imaged, c) obtaining image data set, and d) displaying the image data set.

The term “image” and the term “imaging” can refer to a variety of information outputs and associated techniques for gathering useful information from administered nanoparticles. For example, in one form of basic imaging, spectroscopy is employed as an imaging technique to derive a general determination as to whether the concentration of nanoparticles has increased in a localized region. This is indicative of the presence of a malignancy technique for determining the presence of a malignant tumor in the body. After such a determination, further imaging can be undertaken (either contemporaneously or after a predetermined time interval) to determine the precise characteristics, size, shape, type, etc. of the area/tissue. This further imaging can employ different and/or more-sensitive imaging devices than those initially employed on the localized areas of nanoparticles. These further imaging devices may or may not be particularly sensitive to nanoparticles. Such devices include, but are not limited to MRIs, etc. as described herein. The terms “image” and “imaging” are, therefore, expressly meant to include all types of external scanning mechanisms for localizing nanoparticles.

In certain embodiments, the disclosure contemplates using nanoparticles disclosed herein for magnetic resonance imaging (MRI). In certain embodiments, the disclosure relates to methods of MRI comprising: a) administering nanoparticles as in disclosed herein to a subject, b) applying a magnetic field and radio frequency energy to a region of the subject to be imaged, c) obtaining a magnetic resonance signal image data set, and d) displaying the image data set; wherein the magnetic resonance signal image data set is associated with distribution of the nanoparticles in the region. In certain embodiments, the methods optionally further comprises administering a contrast agent, e.g., comprising Gd³⁺. In certain embodiments, the nanoparticles are iron oxide or iron oxide nanoparticles comprising Gd.

In certain embodiments, the region is the organs of the chest and/or abdomen such as the heart, liver, biliary tract, kidneys, spleen, bowel, pancreas, and adrenal glands. In certain embodiments, the region is the pelvic organs including the bladder and the reproductive organs such as the breast, uterus and ovaries in females and the prostate gland in males. In certain embodiments, the region is blood vessels, brain, and brain stem. In certain embodiments, the region is the lymph nodes. In certain embodiments the method is done to aid in the diagnoses or monitor treatment for conditions such as tumors of the chest, abdomen or pelvis; diseases of the liver, such as cirrhosis, and abnormalities of the bile ducts and pancreas; inflammatory bowel disease such as Crohn's disease and ulcerative colitis; heart problems, such as congenital heart disease; malformations of the blood vessels and inflammation of the vessels (vasculitis); or a fetus in the womb of a pregnant woman.

In certain embodiments, the region is the kidney wherein the subject is diagnosed with nephrogenic systemic fibrosis.

Diffusion imaging is an MRI method that produces in vivo magnetic resonance images of biological tissues sensitized with the local characteristics of molecular diffusion. In a typical T₁-weighted image, nuclei in a sample are excited with the imposition of a magnetic field. This causes many of the nuclei to precess simultaneously, producing signals in MRI. In T₂-weighted images, contrast is produced by measuring the loss of coherence or synchrony between the nuclei. When nuclei are in an environment where they can freely tumble, relaxation tends to take longer. In certain clinical situations, this can generate contrast between an area of pathology and the surrounding healthy tissue.

In certain embodiments, the disclosure contemplates using nanoparticles disclosed herein for diffusion magnetic resonance angiography (MRA). Generating images of blood vessels, both arteries and veins, may be based on flow effects or on contrast (inherent or pharmacologically generated). In certain embodiments, nanoparticles disclosed herein are used as intravenous contrast agents.

In certain embodiments, the disclosure contemplates using nanoparticles disclosed herein for real-time MRI, i.e., to the continuous monitoring (filming) of moving objects in real time. In certain embodiments, the disclosure contemplates using nanoparticles disclosed herein for the images produced by an MRI scanner are used to guide minimally invasive procedures or concurrent with a surgical procedure or a surgical procedure is temporarily interrupted so that images can be acquired to verify the success of the procedure or guide subsequent surgical work.

In certain embodiments, nanoparticles reported herein may be used in applications in diagnosis and treatment of certain cancers. An infusion via, for example, injection of a compound containing nanoparticles allows for imaging to be used in diagnosis, and where applicable, treatment of certain cancers. Although it is not intended the embodiments of the disclosure be limited by any particular mechanism, it is thought that the injected nanoparticles are absorbed by phagocytes. The phagocytes then collect in the cancer/tumor site, thereby producing an increased concentration of nanoparticles within the cancer site. Advantageously, this allows for detection of tumor masses and cancer cells. The phagocytes (phagocytic leukocytes) are types of cells that can migrate to the site of the malignancy and can be followed by detecting levels of nanoparticle uptake to the cells for imaging to assist in diagnosis.

Detection of ovarian cancer, for example, can be accomplished by following cells in the peritoneum as they collect at the site of the malignancy. Ovarian cancer is initially restricted to the peritoneal cavity for imaging and/or treatment purposes. Direct application into the peritoneum avoids the need for systemic nanoparticle delivery required in other forms of cancer, and bypasses the problem of nanoparticle sequestration in the lung and liver. Therefore, intraperitoneally administered nanomaterials, including iron oxide nanoparticles as described herein, are absorbed by the phagocytes. However, while the illustrative procedure is applied to organs and tissues where nanoparticles are not generally free to migrate throughout the body, it is expressly contemplated that the techniques herein can be adapted for use in a variety of organs using appropriate mechanisms to concentrate or control the migration of nanoparticles, so that targeted regions are more particularly provided with nanoparticle populations.

Several methods of detecting the levels of nanoparticles collected at the malignancy can be employed including but not limited to MRI, optical imaging, nuclear medicine and nanoparticle spectroscopy.

In certain embodiments, this disclosure relates to using nanoparticles disclosed herein for diagnosis. The nanoparticles may be selectively delivered proximate a cancer site containing cells associated with the cancer. This can be performed, for example, by directly administering a nanoparticle agent into a patient using a needle. Other techniques for administering the nanoparticle agent are apparent to those of ordinary skill. The concentration of nanoparticles administered in the agent is variable depending upon the sensitivity of the apparatus being employed, the treatment being performed, and other factors apparent to those of ordinary skill.

After the nanoparticle agent has been administered, a predetermined time period lapses to allow for uptake of the nanoparticles to the cancer cells. The predetermined time for waiting for nanoparticle uptake is highly variable depending upon the type of cancer and concentration of nanoparticles within the nanoparticle agent and can vary.

In certain embodiments, the disclosure contemplates treatment using nanoparticles disclosed herein. A nanoparticle-based agent administered as described herein may be further used in the treatment of cancer cells. The procedure provides for by directing an agent containing nanoparticles to be selectively delivered to a cancer site. The cancer site contains cells associated with a cancer, which selectively uptake the nanoparticles delivered in the agent. After a predetermined time period is specified to allow for uptake of the nanoparticles into the cancer cells, a field is applied to the nanoparticles for a sufficient period of time to activate the magnetic cores of the nanoparticles. This thereby induces hyperthermia-mediated destruction of the cells by heating up the nanoparticles, and more particularly the magnetic cores of the nanoparticles. The cancer cells are specifically targeted, as they uptake higher concentrations of the nanoparticles. Accordingly, the cancer cells that have taken up the nanoparticles can be destroyed by thereafter heating the nanoparticles.

### Magnetic Resonance Imaging (MRI)

Atomic isotopes that contain an odd number of protons, such as the hydrogen nuclei in water molecules, spin and generate magnetic fields (magnetic moment). Ferromagnetic materials, e.g., iron, lanthanide elements such as gadolinium, or Gd, and materials with magnetic moments can be used in magnetic resonance imaging (MRI) where an external uniform magnetic field is applied to a region of the subject to be imaged. Radio frequency (RF) energy is applied to this region. The RF energy creates a magnetic field that excites the magnetic material within the tissues of the subject, e.g., the hydrogen nuclei in water molecules. The excited nuclei spin emit RF signals, referred to herein as magnetic resonance signals. By applying magnetic field gradients so that the magnitude of the magnetic field varies with location within the body of the subject, the magnetic resonance phenomenon can be limited to only a particular region or “slice” of the body, so that all of the magnetic resonance signals come from that slice. Moreover, by applying additional magnetic field gradients, the frequency and phase of the magnetic resonance signals from different locations within the slice can be made to vary in a predictable manner depending upon the position within the slice. Stated another way, the magnetic resonance signals are “spatially encoded,” so that it is possible to distinguish between signals from different parts of a slice.

If this process is repeated numerous times to elicit signals using different gradients, it is possible to derive a set of information which indicates one or more characteristics of magnetic resonance signals from particular locations within the body of the subject. Such a set of information is referred herein to as an image data set. Because the characteristics of the magnetic resonance signals vary with the concentration of different chemical substances and other chemical characteristics of the tissues, different tissues provide different magnetic resonance signal characteristics. When a magnetic resonance signal image data set is displayed in a visual format, such as on a computer screen or printed image, the information forms a picture of the structures within the body of the subject, with different tissues having different intensities or colors.

Typically, a magnetic resonance image data set is stored as a set of individual data elements. The data in each element represents one or more characteristics of magnetic resonance signals from a small volume element or “voxel.” For example, the map can be stored as a three-dimensional array of data elements, the dimensions of the array corresponding to three-dimensional space. Data elements corresponding to a given plane in three-dimensional space can be selected for display in a two-dimensional picture such as a screen display or printed image. Each small area element on the surface of the picture, commonly referred to as a “pixel,” is assigned an intensity or color value based on the numerical values of the data element for the corresponding voxel.

Image contrast may be weighted to demonstrate different anatomical structures or pathologies. Each tissue returns to its equilibrium state after excitation by the independent processes of T₁ (spin-lattice) and T₂ (spin-spin) relaxation. To create a T₁-weighted image magnetization is allowed to recover before measuring the MR signal by changing the repetition time (TR). To create a T₂-weighted image magnetization is allowed to decay before measuring the MR signal by changing the echo time (TE).

Magnetic resonance imaging can show abnormal tissues in contrast to surrounding normal tissues. For example, magnetic resonance signals from malignant tumors have a characteristic referred to as the spin-lattice relaxation time or “T₁” different from the T₁ of normal tissues. If a magnetic resonance image is taken so that the data in each data element depends at least in part on the T₁ of the tissue at the corresponding location, a picture showing malignant tumor tissue in contrast to normal tissue can be displayed.

Pictures derived from MRI images are typically read by a physician visually examining the picture to diagnose disease which may be present or to evaluate the progress of a known disease. Such evaluation may involve, for example, a mental comparison by the physician with pictures the physician has previously seen of normal and other diseased subjects or pictures taken in the past of the same subject. In this process, the physician typically attempts to discern the outlines of body structures in the picture. This may also be an automated process in which the computer examines a new image to extract “features relating to particular disease states” using a pattern recognition technique and stores signals descriptive of these features in a “fact database.” These “feature signals” are compared with similar “feature signals” extracted from previously acquired images and the resulting comparison information is subjected to artificial intelligence rules to provide “a diagnostic assessment.”

Typically the MRI apparatus includes gradient coils, a transmitter and a receiver, which themselves may be of the conventional types used in magnetic resonance imaging apparatus, as well as a system controller linked to the gradient coils, transmitter and receiver, the system controller being operative to actuate the gradient coils, transmitter and receiver to perform the sequence of operations required to elicit magnetic resonant signals from a subject in the subject receiving space, commonly referred to as a “pulse sequence.” Most desirably, the support controller is operatively associated with the system controller so that magnetic resonance signals are elicited. Typically the apparatus can automatically move the subject and automatically acquire magnetic resonance data sets in a variety of subject positions. Magnetic resonance imaging includes the steps of automatically moving the subject through a pre-selected sequence of dispositions relative to a static field magnet, e.g., without moving parts of the subject relative to one another. The moving step is performed so that in at least a plurality of the pre-selected dispositions the subject is subjected to a static field provided by the static field magnet. Magnetic resonance signals are elicited from the subject in the plurality of pre-selected dispositions.

As mentioned above, use of the comparison image data set in a visual display, either by display of the comparison image data set itself or by using the comparison image data set to highlight a visually-perceptible image generated from another data set allows use of the comparative data without reliance on artificial intelligence or automated pattern recognition schemes. However, such schemes may be applied to the image data set. Merely by way of example, the system may find the dimensions of any region of contiguous voxels in the comparison image data set having non-zero values, or values above a selected threshold. Similarly, the comparison image data set can be processed to provide additional information. For example, the comparison image data set may be subjected to an automated feature-extraction process to find characteristics of the comparison image data set or of particular portions of the difference, features such as the ratio of dimensions can be found for each such region. These and other features can be extracted and compared with known disease patterns either in a rule-based system or by a neural network or other system capable of learning by exposure to a known learning set of comparison images.

The magnetic resonance data can in one or more of the image data sets can be examined automatically to select data elements corresponding to one or more particular tissue types, and the highlighting step can be limited to regions of the displayed image corresponding to particular tissue types. For example, in studies of the spine, only those pixels corresponding to voxels including disc tissue may be highlighted.

While the discussion above refers to gathering information for the voxels within a single slice of tissue, each image data set typically includes information for voxels in numerous slices, and hence each image data set includes information for voxels in a three-dimensional array of voxels.

MRI can be applied using a static field magnet incorporating physical poles aligned on a polar axis, and the field surrounds the polar axis or applied using other types of magnets as, for example, magnets which use electromagnet coils to form the static magnetic field. For example, certain magnets can include a set of superconducting main field coils held in spaced-apart relationship to one another so as to provide a subject receiving space between them. Auxiliary or “bucking” coils may be provided in association with the main field coils. The coils are arrayed along a horizontal coil axis or field axis. In known manner, the numbers of turns in the coils, the sense of the current flowing within such turns and the magnitude of such currents or selected so as to provide a substantially uniform field directed along the field axis within an imaging region or inside the subject receiving space and to suppress “fringe” fields outside of the subject receiving space. Such a magnet typically does not incorporate a ferromagnetic frame or physical poles. Such a magnet can provide a field of similar orientation and configuration to the magnets discussed above. The term “field axis” as used herein refers generally to an axis such that the field vector of the static magnetic field is parallel to the axis, and the field surrounds the axis. Stated another way, the field axis extends through the subject-receiving space of the magnet. In a magnet having poles, the field axis typically is coincident with the polar axis.

### Contrast agents

Iron oxide nanoparticles (IONPs) contain maghemite and magnetite and are ferromagnetic. The techniques of hydrogen nuclei in water can be applied to ferromagnetic contrast agents, e.g., the IONPs reported herein, so as to monitor changes in the shape of a body cavity filled with the agents. Where a contrast agent is employed, the comparison image data set can be revised to eliminate voxels which do not contain the contrast agent, and thereby limit highlighting in the displayed image to only pixels showing the contrast agent. In general, iron oxide nanoparticles require stabilization in order to prevent aggregation. IONPs with core sizes larger than 10 nm can lead to so-called T2 contrast, or signal darkening or hypointensity.

Gadolinium (III) containing MRI contrast agents are typically used for enhancement of vessels in MR angiography or for brain tumor enhancement, e.g., associated with the degradation of the blood-brain barrier. Gd(III) chelates do not pass the blood-brain barrier because they are hydrophilic. Thus, these are useful in enhancing lesions and tumors where the Gd(III) leaks out. Contemplated agents include gadoterate, gadodiamide, gadobenate, gadopentetate, gadoteridol, gadofosveset, gadoversetamide, gadoxetate, and gadobutrol. In certain embodiments, the disclosure contemplates that the nanoparticles reported herein, e.g., IONPs reported herein, can be used instead of or in combination with the gadolinium(III) containing MRI contrast agents in such methods. For example, contrast agent(s) are injected into a tumor or a vein. A magnetic resonance angiography is then obtained to look at the amount of blood going through different parts of the brain and/or tumor. In contrast with IONPs, gadolinium(III) based agents cause mostly T₁ contrast, or signal increasing or hyperintensity, in the affected anatomic region or abnormal tissues.

### Conjugation with Other Surface Molecules

In certain embodiments, the disclosure contemplates further conjugation of specific binding agents to the saccharide coated nanoparticles disclosed herein. Lysine and polypeptides such as proteins, antibodies, and fragments, can be covalently linked to the saccharide coating of the nanoparticles using a periodate-oxidation/borohydride-reduction method, which, through the formation of Schiff bases as intermediates, covalently links the amine (lysine) groups of the protein to the alcohol groups of the saccharide coating. Alternative ways of attaching surface molecules include glutaraldehyde crosslinking, using the biotin-streptavidin system and amine-sulfhydryl group linkage.

By including a molecule containing a chemical group, amenable to functionalization during thermal synthesis e.g., diethylene glycol amine, serine, or glucose containing an amine, carbocyclic acid, or thiol groups, one can conjugate other surface molecules to the nanoparticles disclosed herein. Animated saccharides may be made by substituting or adding an amine containing molecules or amine containing saccharides such as glucosamine or galactosamine in place of or in addition to glucose, during the thermal synthesis methods disclosed herein. The presence of aminated molecules of saccharides on the outer surface represents a suitable platform for further conjugation to specific binding agents and biomolecules.

In certain embodiments, contemplated surface molecules for conjugation included nucleotides, oligonucleotides, amino acids, polypeptides, ligands, receptors, drugs, steroids, antibodies, and antibody mimetics. In certain embodiments, the disclosure contemplates saccharide coated nanoparticles disclosed herein covalently linked to antibodies, binding antibody fragments, specific binding agents, cholescytokinin, secretin, transferrin, synaptotogmin I protein, and human amyloid beta-protein (Abeta) 1-40.

Specific binding agents such as antibodies and antibody fragments that specifically bind to cancer antigens, e.g., HER-2, are within the scope of the present disclosure. The antibodies may be polyclonal including mono-specific polyclonal, monoclonal (mAbs), recombinant, chimeric, single chain, multi-specific and/or bi-specific, as well as antigen-binding fragments, variants, and/or derivatives thereof.

Numerous methods known to those skilled in the art are available for obtaining antibodies or antigen-binding fragments thereof. For example, antibodies can be produced using recombinant DNA methods (U.S. Pat. No. 4,816,567). Monoclonal antibodies may also be produced by generation of hybridomas in accordance with known methods. Hybridomas formed in this manner are then screened using standard methods, such as enzyme-linked immunosorbent assay (ELISA) and surface plasmon resonance analysis, to identify one or more hybridomas that produce an antibody that specifically binds with a specified antigen. Any form of the specified antigen may be used as the immunogen, e.g., recombinant antigen, naturally occurring forms, any variants or fragments thereof, as well as antigenic peptide thereof.

The modular structure of antibodies makes it possible to remove constant domains in order to reduce size and still retain antigen binding specificity. Engineered antibody fragments allow one to create antibody libraries. A single-chain antibody (scFv) is an antibody fragment where the variable domains of the heavy (VH) and light chains (VL) are combined with a flexible polypeptide linker. The scFv and Fab fragments are both monovalent binders but they can be engineered into multivalent binders to gain avidity effects. One exemplary method of making antibodies and fragments includes screening protein expression libraries, e.g., phage or ribosome display libraries. Phage display is described, for example, in U.S. Pat. No. 5,223,409.

In addition to the use of display libraries, the specified antigen, e.g., 8 to 10 amino acid segment of a cancer antigen optionally fused to another polypeptide, can be used to immunize an animal, typically a rodent, e.g., a mouse, hamster, or rat. Using the hybridoma technology, antigen-specific monoclonal antibodies derived from the genes with the desired specificity may be produced and selected. U.S. Pat. No. 7,064,244.

Antibody mimetics or engineered affinity proteins are polypeptide based targeting moieties that can specifically bind to targets but are not specifically derived from antibody VH and VL sequences. Typically, a protein motif is recognized to be conserved among a number of proteins. One can artificially create libraries of these polypeptides with amino acid diversity and screen them for binding to targets through phage, yeast, bacterial display systems, cell-free selections, and non-display systems. See Gronwall & Stahl, J Biotechnology, 2009, 140 (3-4), 254-269, hereby incorporated by reference in its entirety. Antibody mimetics include affibody molecules, affilins, affitins, anticalins, avimers, darpins, fynomers, kunitz domain peptides, and monobodies.

### EXAMPLES

### Synthesis of Hydrophobic Iron Oxide Nanoparticles (IONPs)

The hydrophobic iron oxide nanoparticles were synthesized by thermo-decomposition. iron(III) oleate was first prepared. Typically, 4.04 g of ferric nitride (10 mmol) and 9.13 g of sodium oleate (30 mmol) was dissolved in the solvent mixed with 40 mL distilled water, 50 mL hexane and 10 mL absolute ethanol. The mixture of iron oleate was stirred at room temperature for 4 hours, and then kept still overnight. The resulting red-brownish hexane layer was used as the iron source for thermo-decomposition. In a typical reaction, 5 mL of the iron oleate was mixed with 5 mL of 1-octadecene at room temperature, and degassed with ultrahigh argon for 20 min. After evaporating hexane at 70 ° C., the reaction mixture was heated to 320° C. with a heating rate of 0.6° C.·s−1. The reaction time was adjusted to control the size of IONPs, which was about 5 min for IONPs with a core size of 3.5 nm, and reheated approximate 10, 15, 20, 30 min for IONPs with 4.8, 9.9, 15.6, 19.9 nm core size. After cooling down to room temperature, ethanol was added to precipitate the nanoparticles. The products were collected by centrifugation, and washed with hexane and ethanol for several times.

### Synthesis of Oligosaccharide Coated Iron Oxide (SIO) Nanoparticles

Oligosaccharide coating was introduced on the hydrophobic IONPs by in situ-polymerization. Briefly, the oleic acid coated IONPs were redispersed in chloroform after purified with centrifugation, and carefully added dropwise into the preheated glucose solution in dimethylformamide (DMF). The mixture was heated to 120° C., and kept at this temperature for 2.5 hours. After cooling down to room temperature, the product was precipitated by adding ethanol. The precipitant was washed and centrifuged several times. The final product was collected and redispersed in distilled water for other characterization and applications.

### Characterizations of SIO Nanoparticles

The morphology and size of SIO nanoparticles were studied using transmission electron microscope (TEM, Hitachi H-7500, accelerating voltage 75 kV). Typically, TEM samples are prepared by dropping diluted nanoparticle solutions on the carbon coated copper grid and air-dried. The hydrodynamic size and surface charges of nanoparticles in the aqueous solution were evaluated using a dynamic light scattering (DLS) instrument (Malvern Zeta Sizer Nano S-90) equipped with a 22 mW He—Ne laser operating at 632.8 nm. The structural analysis of SIO nanoparticles was carried out by powder X-ray diffraction (XRD, Bruker D8 DIFFRAC powder diffractometer, Co Kα). For studying the nanoparticles coating, Fourier transform infrared spectroscopy (FTIR) spectra were collected on a PerkinElmer Spectrum 100 FT-IR spectrometer (Bucks, UK). UV-vis absorption spectra were obtained with a scanning spectrophotometer (Shimadzu UV-2401PC) with a slit width of 1.0 nm.

### Measurement of Relaxation Times and Calculation of Relaxivities

To evaluate MRI contrast enhancement capability, SIO solutions with different concentrations were examined with a 3T MRI scanner (Magnetom Tim Trio, Siemens Medical Solutions, Erlangen, Germany) using T₁- and T₂-weighted fast spin echo sequences, an inversion recovery turbo spin echo sequence and a multi-echo T₂-weighted spin echo sequence. A commercial T1 contrast enhancement agent Multihance® (Gd-BOPTA) was used for comparing the MRI contrast enhancement effect. Each sample was prepared with Fe or Gd concentrations varying from 0.004 to 40 mM. To measure the longitudinal relaxation time T₁, an inversion recovery turbo spin echo (TSE) sequence with an echo train length (ETL) of 3, an echo time (TE) of 13 ms and a repetition time (TR) of 1500 ms was used to obtain images at different inversion times (TI) of 23, 46, 92, 184, 368, 650, 850, 1100, and 1400 ms, respectively. To measure the transverse relaxation time T₂, a multi-echo spin echo sequence was used with a TR of 2400 ms and 15 TEs, starting at 11 ms with increments of 11 ms. The signal intensity (SI) of each region-of-interest (ROI) at different TIs or TEs was measured for samples of each concentration.

### MRI of Mice Administered with SIO Nanoparticles

BALB/c mice were anesthetized by intraperitoneal injection of a ketamine-xylazine mixture (95:5 mg kg⁻¹). The saline diluted SIO-3 solution was intravenously administered at a dosage of 2.5 and 10 mg Fe per kg of mouse body weight. For comparison, Gd-BOPTA and SIO-20 (core size of 20 nm) were injected at the dosage of 2.5 mg kg⁻¹ and 0.2 mmol kg⁻¹, respectively. Fat suppressed T₁-weighted spin echo images were obtained to investigate the contrast changes in different organs and anatomic structures, such as the liver, kidney and iliac artery, at different time points. The imaging parameters included: TR=724 ms, TE=10 ms, matrix=320×134, field of view (FOV)=120×60 mm², flip angle=70, and slice thickness=1.00 mm. The signal-to-noise ratio (SNR) was calculated according to the equation: SNR=SImean/SDnoise. The relative contrast enhancement at different time points was defined as the signal decrease ΔSNR=(SNRpre−SNRpost)/SNRpre. The contrast-to-noise ratio between the liver parenchyma and vasculature was calculated as CNR=(SNRpost(vasculature)−SNRpost(liver parenchyma))/SNRpre(liver parenchyma).

### Body Clearance of SIO Nanoparticles in Mice

The clearance of nanoparticles was evaluated by both chemical analysis of iron contents from the collected organs or tissues and ROI analysis of T₂-weighted MRI and T₂ relaxometry mapping of live animals, which allows for time dependent changes of iron concentrations at the specific organ in the same animal. SIO-3, SIO-20 and SHP20, which are commercially available amphiphilic polymer coated SPIO nanoparticles (core size 20 nm from Ocean NanoTech, LLC), were intravenously administered into BALB/c mice (n=3) at a dosage of 2.5 mg kg⁻¹ mouse weight. For MRI monitoring, T₂-weighted MR images of the mice were acquired on a 3T MRI scanner before and after administration of nanoparticle contrast agents using a volumetric wrist coil. The imaging parameters included: TR=3710 ms, TE=12-180 ms, matrix=256×128, field of view (FOV)=120×60 mm², flip angle=180° and slice thickness=1 mm. Colorized T₂ maps were then generated as described in the ESI. ROIs with the same areas were drawn in the liver and spleen at the same T₂ maps. The relative contrast enhancement at different time points was calculated to show the average signal changes. The organs (liver, spleen, kidney, lungs, heart, and muscle) and blood samples were collected at 10 min, 1 day, 1 week, 2 weeks and 3 weeks after injection. For chemical analysis of tissue iron, the phenanthroline colorimetric method was used to determine the iron concentration in organs after the organs were digested in concentrated HNO₃. In addition, Prussian blue staining was performed for the major organ slices following a standard protocol. Briefly, frozen tissues mounted in an optimal cutting temperature compound (OCT) were sliced in 8 μm thickness, fixed with 4% paraformalin for 10 min, then soaked into the working solution composed of 10% potassium ferrocyanide(II) trihydrate and 20% HCl solution (v:v=1:1) at 37° C. for 4 hours. After being washed with PBS, the slices were counterstained with nuclear fast red for 5 min. Blue dots representing the remaining IONPs in organs were investigated with a light microscope.

### Hydrophobic IONPs

The heating rate and the decomposition time were precisely adjusted to prepare different sized IONPs prepared by thermal decomposition of ferric oleate. The core sizes of obtained hydrophobic IONPs were highly uniform with diameters of 3.5±0.5 nm (SIO-3) and 19.9±1.6 nm (SIO-20), respectively. After in situ-polymerization of glucose on the surface of hydrophobic IONPs, a coating layer of oligosaccharides was formed. The prepared nanoparticles become water soluble with core sizes unchanged as shown in TEM images. The oligosaccharides coated iron oxide nanoparticles showed single dispersed in the aqueous solution revealed by the TEM and dynamic light scattering (DLS) measurement. The hydrodynamic diameters of oligosaccharide coated IONPs, i.e., SIO-3 (3.5 nm core size) and SIO-20 (20 nm core size), are 7.3 nm and 20.9 nm, respectively, as determined by DLS. These oligosaccharides coated nanoparticles were water-soluble and highly stable in the aqueous media and room temperature for at least 2 months. Both SIO-3 and SIO-20 showed an X-ray diffraction (XRD) pattern that can be indexed to the cubic spinel structured magnetite or maghemite. XRD revealed that the crystallinity and grain size also remain unchanged after applying the surface coating.

### Intravenously Administering IONPs

When administering IONPs in mice intravenously at both low (2.5 mg Fe/kg) and high (10 mg Fe/kg) dosages, IONPs showed excellent T₁ contrast enhancement in the kidney and iliac artery that is similar to that observed in Gd-BOPTA enhanced MRI. In comparison, T₁ contrast enhancement is not obvious when using SIO-20 with a larger core size. Interestingly, SIO-3, particularly at the higher dosage, led to the “dual” contrast effect in liver with T2 “darkening” in the normal liver tissue but “bright” T₁ contrast in the hepatic vasculature. The observed T₂ contrast in the liver and spleen is attributed to the aggregation of nanoparticles resulted from the uptake of IONPs by RES cells, leading to the increased r₂, while IONPs circulating in the vasculature are highly dispersed, thus continue to provide T₁ contrast. The “dual” contrast effect by SIO-3 observed in liver imaging further improves the image quality for visualizing liver tissue and hepatic vasculature that cannot be achieved by either SIO-20 or Gd-BOPTA. Therefore, it provides the capability of circumscribing a liver mass with information from both size/volume and tumor vasculature at the same time. The T₁ contrast enhancement in kidney by SIO-3 may also offer potential applications of imaging of renal functions, especially in patients suffering from NSF and are vulnerable to Gd toxicity. The accumulation of SIO-3 in the kidney, but not SIO-20, suggested the possible kidney clearance of the sub-5 nm IONPs.

### MRI Contrast Enhancement Effects

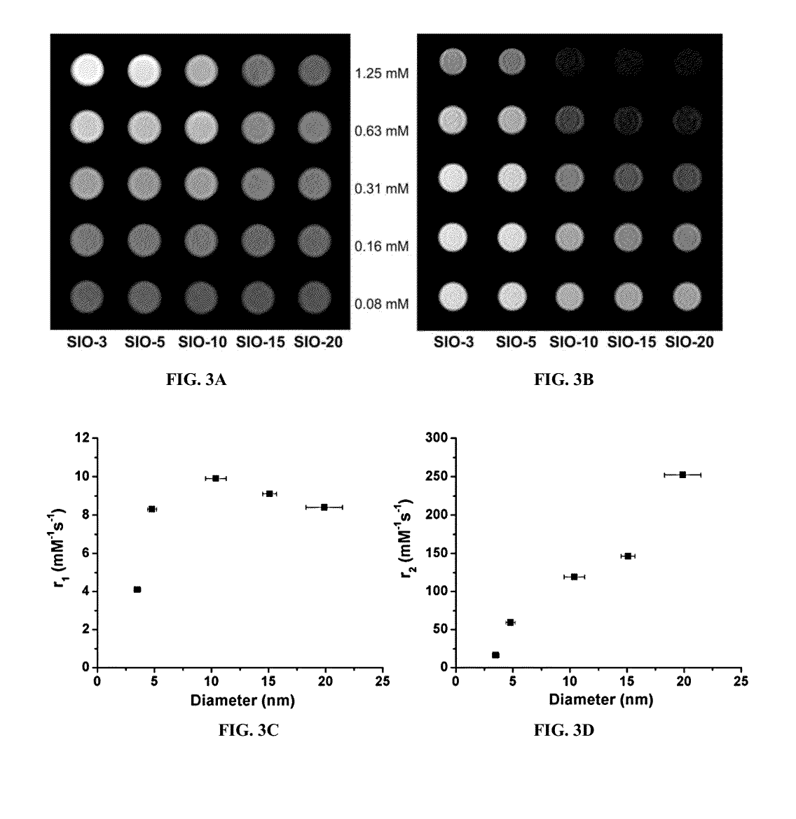

The MRI contrast enhancement effects of SIOs were investigated at the clinically relevant magnetic field (3 T). FIGS. 3A and 3B show T₁- and T₂-weighted MR images of the SIO aqueous solutions with different Fe concentrations. SIO-3 exhibits the highest T₁ contrast enhancement, while SIO-20 exhibits the highest T₂ contrast enhancement. SIO-3 has the highest surface-to-volume ratio due to the ultrafine size. For nanoparticulate contrast agents, the T₁ contrast enhancement is believed to be majorly contributed by the inner-sphere relaxivity, which comes from the direct coordination between water molecules and magnetic ions on the nanoparticle surface. A high surface-to-volume ratio in combination with a thin hydrated coating layer of oligosaccharides for SIO-3 would facilitate the water molecule interaction with the inner layer.

Both r₁ and r₂ of SIO showed size dependency (FIGS. 3C and 3D). The increased r₂ with increased size could be ascribed to size dependent magnetic susceptibility . On the other hand, r₁ of SIOs kept increasing until a maximum was reached at the nanoparticle size around 10 nm. The similar trends were observed for the ultrasmall IONPs coated with PEG, CTAB, and DEG. Such size dependency on r1 is attributed to the monodispersed size, together with a compact and highly hydrophilic coating, resulting in good dispersity in solvent without aggregation. For larger nanoparticles (>10 nm), longitudinal relaxivity r₁ decreased with the increased size, which is attributed to the locked nanoparticle magnetic moment on anisotropy axes, thus relaxivity is dominated by Curie relaxation.

To further evaluate the contrast enhancement efficiency and behavior of SIOs, a signal intensity profile of a contrast agent was computed using the equation describing signal intensity (SI) evolution from the T₁-weighted spin echo sequence. Given the same Fe concentration (i.e., 1 mM) and image acquisition parameters (i.e., TR=500 ms and TE=12 ms) typically used for T₁-weighted spin echo MRI, the highest T₁ contrast, i.e., brightest signal, for a given r₁ can be only obtained when r₂ reaches zero. Furthermore, SI is more sensitive to the change in r₂ than in r₁ for the contrast agents with an r₁ larger than 4.5 mM⁻¹s⁻¹. For example, although SIO-3 has a lower r₁, it has a much higher T₁ enhancement efficiency than SIO-20 because of a sharper reduction in r₂.

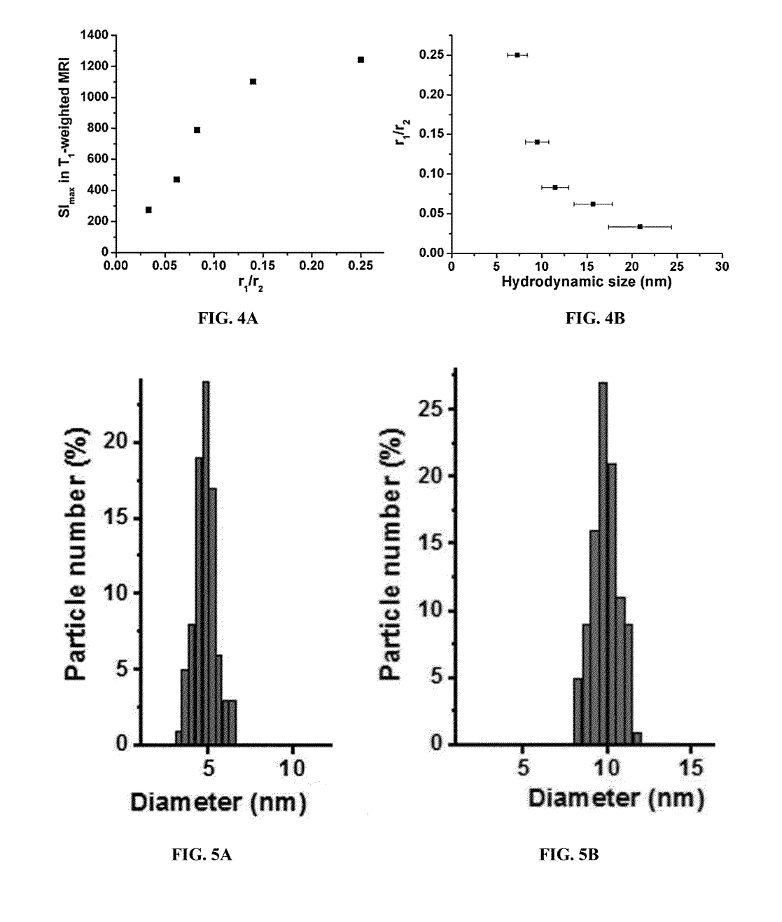

The r₁/r₂ ratio may dictate the T₁ contrast enhancement properties of the magnetic nanoprobes. An increased maximum SI in T₁-weighted MR images was observed with the increasing r₁/r₂ ratio as shown in FIG. 4A. It has been theoretically studied that the r₁/r₂ ratios are monotonically increased against the translational diffusion time τD, which is related to the radius of IONPs, water permeability of the coating layer and the coating thickness. Unsurprisingly, SIO-3 has the highest r₁/r₂ ratio of 0.25 compared to the counterparts in larger sizes (FIG. 4B).

The thermal decomposition of the mixture of iron oleate and gadolinium oleate can produce monodisperse Gd₂O₃-embedded iron oxide (GdIO) nanoparticles. For example, magnetic cations with unpaired electrons (e.g. Mn²⁺, Gd³⁺) have been introduced into iron oxide nanostructures to increase r₁, thus to realize the positive contrast enhancement. See Zhou et al., Adv Mater, 2012, 24 (46): 6223-6228. Gd-doping (made by the process similar to IONP of mixing Mn²⁺, Gd³⁺, and Fe³⁺ salts with sodium oleate) may be a more effective way because of the slighter increase of r₂.

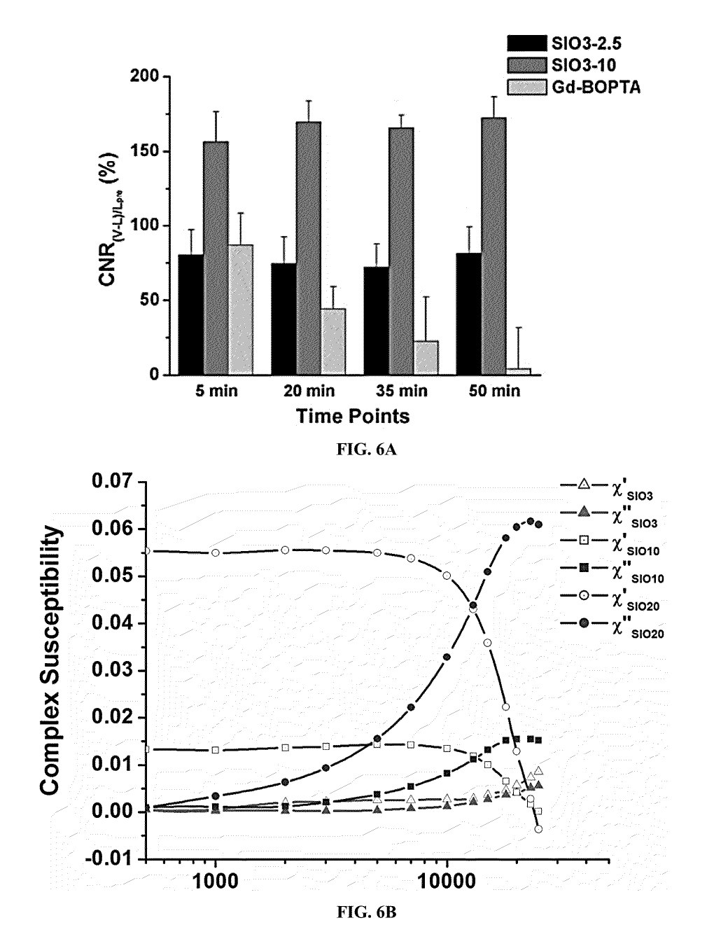

The “dual” contrast effect of SIO-3 improves the sensitivity and image clarity for visualizing the morphology of the liver parenchyma and the structure of hepatic vasculature, which cannot be achieved by either SIO-20 or Gd-BOPTA alone (FIG. 6A). Therefore, it potentially provides the capability of circumscribing a liver mass or detection of very small liver lesion with information from both size/volume and tumor vasculature at the same time using only one contrast agent instead of generating “double” contrast by sequentially injections of both IONPs and Gd-DTPA.

T₁ contrast enhancement in kidney by SIO-3 may also offer potential applications of imaging renal functions, especially in patients suffering from nephrogenic systemic fibrosis (NSF) and who are vulnerable to Gd toxicity. Patients with NSF develop large areas of hardened skin with fibrotic nodules and plaques. NSF may also cause joint contractures resulting in joint pain and limitation in range of motion. In its most severe form, NSF may cause severe systemic fibrosis affecting internal organs including the lungs, heart and liver. Evidence suggests NSF is associated with exposure to gadolinium (with gadolinium-based MRI contrast agents being frequently used as contrast agents for magnetic resonance imaging (MRI)) in patients with severe kidney failure.

The accumulation of SIO-3 in the kidney, but not SIO-20, suggested possible renal clearance of the sub-5 nm IONPs due to the smaller hydrodynamic size than 8 nm. Both T₁ and T₂-weighted images of mice administrated with SIO-3 showed the gradual changes of MRI signals in the bladder over the time, indicating the excretion of SIO-3 from kidney to the bladder at the time of 1 hour after administration and the stability of the nanoparticles in blood stream upon filtrated and secreted by the kidney. Such MRI signal changes were not observed in the bladders of those animals receiving larger sized IONPs, such as SIO-20.

1. A composition comprising iron oxide nanoparticles comprising a saccharide coating of less than 5 nm.

2. The composition of claim 1, wherein the average core size is less than 5 nm with a hydrodynamic size of not greater than 10 nm.

3. The composition of claim 1, wherein the saccharide coating is made by the process of heating the nanoparticles in a mono-, di- or tri-saccharide in dimethylformamide.

4. The composition of claim 3, wherein the mono-saccharide is glucose, galactose, glucosamine, galactosamine, and N-acetylglucoseamine.

5. The composition of claim 3, wherein the heating is at 120 degrees Celsius for 2.5 hours.

6. The composition of claim 3, wherein the heating is above 100 degrees Celsius and below 150 degrees Celsius for more than an hour.

7. The composition of claim 1, wherein the core is made by the process of mixing iron (III) oleate and 1-octadecene and heating the mixture at a rate of 0.6 degrees Celsius per second until reaching 320 degrees Celsius, holding for 5 minutes at 320 degrees Celsius, and thereafter cooling.

8. The composition of claim 1, wherein the core is made by the process of mixing iron (III) oleate and 1-octadecene and heating the mixture at a rate of 0.01-1.0 degrees Celsius per second until reaching 300 to 320 degrees Celsius, holding for 2 to 30 minutes at 300 to 320 degrees Celsius, and thereafter cooling.

9. The composition of claim 7, wherein the iron (III) oleate and 1-octadecene are in a 1:1 mixture by volume mixture.

10. The composition of claim 7, wherein the iron (III) oleate and 1-octadecene are in a 0.5-5.0 to 5.0-0.5 by volume mixture.

11. The composition of claim 1, wherein 12% of the nanoparticles have a core size of 3.1 nanometers, 29% of the nanoparticles have a core size of 3.3 nanometer and 24% of the nanoparticles have a core size of 3.6 nanometers and 16% of the nanoparticles have a core size of 3.9 nanometer.

12. The composition of claim 1, wherein 11-14% of the nanoparticles have a core size of 2.9-3.2 nanometers, 27-30% of the nanoparticles have a core size of 3.2-3.5 nanometer and 23-25% of the nanoparticles have a core size of 3.5-3.8 nanometers and 15 to 18% of the nanoparticles have a core size of 3.8-4.1 nanometer.

13. The composition of claim 1, wherein the core comprises iron oxide and Mn or Gd.

14. A method of magnetic resonance imaging comprising:
a) administering nanoparticles as in claims 1-13 to a subject,
b) applying a magnetic field and radio frequency energy to a region of the subject to be imaged,
c) obtaining a magnetic resonance signal image data set, and
d) displaying the image data set;
wherein the magnetic resonance signal image data set is associated with distribution of the iron oxide nanoparticles in the region.

15. The method of claim 14, wherein the region is the kidney.

16. The method of claim 14, wherein the subject is diagnosed with nephrogenic systemic fibrosis.
